(19) United States
(12) Patent Application Publication
(10) Pub. No.: US 2025/0017590 A1
(43) Pub. Date: Jan. 16, 2025

(54) LEFT-ATRIAL APPENDAGE OCCLUSION

(71) Applicant: Medtronic, Inc., Minneapolis, MN (US)

(72) Inventors: Gabrielle Marie COLVERT, Santa Ana, CA (US); Brendan Thomas COLVERT, Santa Ana, CA (US); Kyle J. DORMAN, Santa Ana, CA (US)

(73) Assignee: Medtronic, Inc., Minneapolis, MN (US)

(21) Appl. No.: 18/617,745

(22) Filed: Mar. 27, 2024

Related U.S. Application Data
(60) Provisional application No. 63/512,968, filed on Jul. 11, 2023.

(51) Int. Cl. A61B 17/12; A61B 90/00; A61F 2/24; A61M 25/10

(52) U.S. Cl. CPC A61B 17/12122; A61B 17/12031; A61B 17/12136; A61F 2/2476; A61B 2017/1205; A61B 2090/3933; A61B 2090/3966; A61F 2210/0014; A61M 25/1002; A61M 2025/1054; A61M 2025/1079

(57) ABSTRACT

The present application relates to an occlusion device for occluding a left atrial appendage. The device has an expandable hollow body with an internal volume. The expandable body includes a proximal portion having a first diameter, wherein the proximal portion is arranged to sealingly engage against an opening of the left atrial appendage, a distal portion having a second diameter, wherein the distal portion is arranged to be positioned inside and at least partially fill the left atrial appendage and a central portion extending between the proximal and distal portion, wherein the central portion comprises a waist region with a third diameter. The third diameter of the waist region is smaller than the first diameter of the proximal portion and the second diameter of the distal portion.

### CROSS-REFERENCE TO RELATED APPLICATIONS

This application claims the benefit of U.S. Provisional Patent Application Ser. No. 63/512,968, filed Jul. 11, 2023, the entire content of which is incorporated herein by reference.

### FIELD OF THE INVENTION

The present invention relates to a method of occluding a left atrial appendage and an occlusion device for occluding a left atrial appendage.

### BACKGROUND OF THE INVENTION

The human heart is a four chambered, muscular organ that provides blood circulation through the body during a cardiac cycle. The four main chambers include the right atrium RA and right ventricle RV which supplies the pulmonary circulation, and the left atrium LA and left ventricle LV which supplies oxygenated blood received from the lungs into systemic circulation. To ensure that blood flows in one direction through the heart, atrioventricular valves (tricuspid valves TV and mitral valves MV) are present between the junctions of the atria and the ventricles, and semi-lunar valves (pulmonary valve and aortic valve) govern the exits of the ventricles leading to the lungs and the rest of the body.

The heart also includes a left atrial appendage LAA, which is a small, car-shaped sac in the muscle wall of the left atrium LA. In normal hearts, when the heart contracts, the blood in the left atrium LA and the left atrial appendage LAA is squeezed out of the left atrium LA and into the left ventricle LV. The LAA has minimal influence on cardiac output and is generally considered to be non-functional structure in the heart.

In atrial fibrillation, an irregular heartbeat causes blood flow to slow enabling clots to form. Because the left atrial appendage LAA is a small sac or pouch, blood may collect there and form clots. The clots often cause thromboembolic complications and risk to the patient. This risk increases if the thrombus or fragments of the thrombus dislodge. If all or a portion of the thrombus flows downstream, it is highly likely that the free material will become trapped in smaller and more tortuous anatomy. This increases a patient's risk for cerebral stroke or peripheral embolism. Thus, in some cases, it may be desirable to exclude or occlude the left atrial appendage LAA such that clots do not form in the left atrial appendage LAA, and if they do, they cannot escape the left atrial appendage LAA. While medications can be taken to control blood clotting and decrease the chance of stroke, a proportion of patients cannot take such medications or follow the strict regimen necessary for continued protection.

To reduce the above-mentioned risks, it is desirable to close the left-atrial appendage to reduce the occurrence of thrombus formation and the risk of thromboembolism. The present disclosure relates to improvements in catheter-based occlusion systems for occluding or excluding the left atrial appendage.

### SUMMARY OF THE INVENTION

A first aspect of the invention provides an occlusion device for occluding a left atrial appendage, the device comprising an expandable hollow body with an internal volume, wherein the expandable body comprises a proximal portion having a first diameter, wherein the proximal portion is arranged to sealingly engage against an opening of the left atrial appendage, a distal portion having a second diameter, wherein the distal portion is arranged to be positioned inside and at least partially fill the left atrial appendage, and a central portion extending between the proximal and distal portion, wherein the central portion comprises a waist region with a third diameter, wherein the third diameter of the waist region is smaller than the first diameter of the proximal portion and the second diameter of the distal portion. Optionally, the central portion is formed from a woven material.

Optionally, the woven material comprises a synthetic polymeric material, such as polyetheretherketone (PEEK).

Optionally, the occlusion device is expandable by the introduction of a fluid into the internal volume of the hollow body.

Optionally, the fluid is a biogel.

Optionally, the biogel comprises radiopaque markers.

Optionally, the central portion comprises a plurality of reinforcement struts that are arranged to strengthen the central portion.

Optionally, the central portion is substantially cylindrical, and the plurality of reinforcement struts extend circumferentially around the central portion.

Optionally, the plurality of reinforcement struts comprises nitinol.

Optionally, the reinforcement strut comprises nitinol wire.

Optionally, the central portion further comprises at least one barb, wherein the at least one barb is arranged to engage with the left atrial appendage to secure the central portion to the left atrial appendage.

Optionally, the central portion is at least partially coated with a drug coating.

Optionally, the distal portion is formed from a woven material.

Optionally, the proximal portion is formed from a woven material.

Optionally, the woven material that forms the distal portion has different characteristics than the woven material that forms the central portion.

Optionally, the woven material that forms the proximal portion has the same characteristics as the woven material that forms the distal portion.

Optionally, the central portion comprises a proximal end and a distal end, and wherein

Optionally, the central portion further comprises at least one circumferential band that at least partially extends around the proximal end of the central portion.

Optionally, the central portion further comprises at least one circumferential band that at least partially extends around the distal end of the central portion.

Optionally, the circumferential bands are formed on the woven material and at least partially comprise radiopaque material.

Optionally, the occlusion device further comprises a third circumferential band extending at least partially around the first diameter of the proximal portion of the occlusion device.

Optionally, when the central portion comprises at least one band at the proximal end and at least one band at the distal end, both circumferential bands are substantially symmetrical.

Optionally, the radiopaque material comprises radiopaque ink.

Optionally, the distal portion is substantially domed.

Optionally, the occlusion device further comprises a central lumen extending from a proximal opening in the proximal portion to a distal opening at a distal end of the distal portion, and at least one aperture extending from the central lumen, wherein the at least one aperture is in fluid communication with the internal volume of the expandable hollow body of the occlusion device.

Optionally, the occlusion device comprises a closure that is moveable between a first and second position; wherein in a first position, the closure prevents flow of fluid in and out of the central lumen through the proximal opening; and in a second position, the closure permits flow of fluid in and out of the central lumen through the proximal opening.

Optionally, the closure device comprises a plurality of leaflets, and wherein the leaflets are moveable between the first and second position.

Optionally, the plurality of leaflets comprise nitinol.

A further aspect of the invention provides a method of occluding a left atrial appendage using a catheter with a central lumen extending from a proximal to distal end, wherein the catheter comprises a deployable sheath containing an occlusion device at the distal end of the catheter, wherein the occlusion device comprises an expandable hollow body with an internal volume, and the expandable body comprises a proximal portion having a first diameter, and the proximal portion is arranged to sealingly engage against an opening of the left atrial appendage; a distal portion having a second diameter, wherein the distal portion is arranged to be positioned inside and at least partially fill the left atrial appendage; and a central portion extending between the proximal and distal portion, wherein the central portion comprises a waist region with a third diameter, wherein the third diameter of the waist region is smaller than the first diameter of the proximal portion and the second diameter of the distal portion, wherein the occlusion device is arranged to move from a collapsed state to an expanded state; the method comprising providing a catheter with the occlusion device in a collapsed configuration; advancing the distal end of the catheter with the device towards an opening of the left atrial appendage; unsheathing the occlusion device to partially expand the occlusion device; advancing the distal portion of the occlusion device into the left atrial appendage; positioning the central portion of the occlusion device in the opening of the left atrial appendage; and inflating the occlusion device into an expanded state.

Optionally, when the occlusion device further comprises a central lumen extending from a proximal opening in the proximal portion to a distal opening at a distal end of the distal portion, and at least one aperture extending from the central lumen, and wherein the at least one aperture is in fluid communication with an internal volume of the occlusion device, the method of inflating the occlusion device into an expanded state comprises inserting the distal end of the catheter into the central lumen of the occlusion device and; delivering a fluid through the at least one aperture and into the internal volume of the occlusion device.

Optionally, the occlusion device further comprises a closure that is moveable between a first and second position; wherein in a first position, the closure prevents flow of fluid in and out of the central lumen through the proximal opening; and in a second position, the closure permits flow of fluid in and out of the central lumen through the proximal opening, wherein inserting the distal end of the catheter into the central lumen moves the closure into second position and the method further comprises removing the distal end of the catheter from the central lumen after fluid has been delivered into the internal volume of the occlusion device and the closure returns to the first position.

Optionally, when the central portion comprises a proximal end and a distal end, and the central portion further comprises at least one circumferential band that at least partially extends around the distal end of the central portion, the method further comprising delivering the fluid into the internal volume of the occlusion device until the fluid reaches the at least one circumferential band.

Optionally, the central portion further comprises at least one circumferential band that at least partially extends around the proximal end of the central portion, the method further comprises delivering fluid into the internal volume of the occlusion device until the fluid reaches the circumferential band that at least partially extends around the proximal end.

Optionally, the occlusion device further comprises a third circumferential band extending at least partially around the first diameter of the proximal portion of the occlusion device, the method further comprises delivering fluid into the internal volume of the occlusion device until the fluid reaches the third circumferential band.

Optionally, the fluid is a biogel, and the method further comprises incorporating radiopaque markers into the biogel.

Optionally, the method further comprises retracting the distal end of the catheter proximally from the central lumen of the occlusion device and into a left atrium adjacent to the left atrial appendage; and delivering dye through the catheter proximally to the occlusion device.

### BRIEF DESCRIPTION OF THE DRAWINGS

Embodiments of the invention will now be described with reference to the accompanying drawings, in which:

FIG. 1 is a schematic sectional illustration of a heart;

FIG. 2 illustrates a diagrammatic view of a patient undergoing a cardiovascular procedure to occlude a left atrial appendage;

FIG. 3A illustrates a distal end of an outer catheter with the occlusion device in a collapsed state;

FIG. 3B illustrates the nested arrangement of the outer catheter and an inner catheter;

FIG. 3C illustrates a cross-sectional view of a capsule arranged at the distal end of the outer and inner catheters;

FIG. 4 illustrates an exemplary left atrial appendage;

FIG. 5 illustrates an exemplary occlusion device in an expanded state;

FIG. 6A-6D are schematic views of an exemplary method of inserting an occlusion device into a heart;

FIG. 7A illustrates the occlusion device in a collapsed state positioned adjacent to a the left atrial appendage;

FIG. 7B illustrates the occlusion device in a collapsed state inserted into the left atrial appendage;

FIG. 7C illustrates the occlusion device in an expanded state in the left atrial appendage;

FIG. 7D illustrates a cross-sectional view of an exemplary occlusion device;

FIG. 7E illustrates a cross-sectional view of the occlusion device in an expanded state in the left atrial appendage;

FIG. 7F illustrates the inner and outer catheters detached from the occlusion device;

FIGS. 8A-8C illustrate an exemplary closure of the occlusion device in an open and closed state;

FIG. 9 illustrates an exemplary method of occluding a left atrial appendage.

### DETAILED DESCRIPTION OF EMBODIMENT(S)

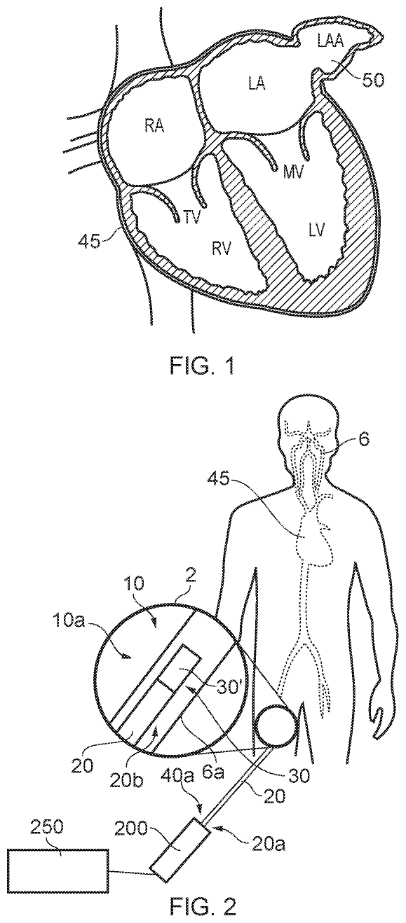

FIG. 1 is a schematic sectional illustration of a human heart 45 that depicts the four heart chambers (right atrium RA, right ventricle RV, left atrium LA, left ventricle LV) and a left atrial appendage LAA 50.

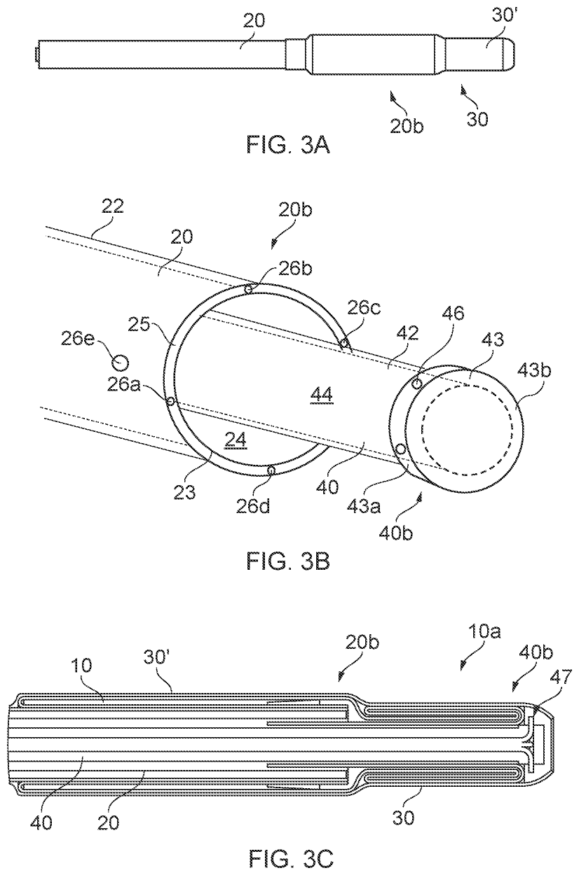

FIG. 2 illustrates a diagrammatic view of a patient undergoing an exemplary left atrial appendage occlusion procedure. As indicated by the enlarged circle area 2, an occlusion device 10 may be inserted into a vasculature 6 of a patient using an outer catheter 20, including for example, into an artery 6a (e.g. a femoral artery) or vein (e.g., femoral vein, internal jugular vein). The occlusion device 10 is held in a collapsed state 10a by a deployable sheath 30. In this example, the deployable sheath 30 is a capsule 30′, however any suitable deployment mechanism may be used to contain the occlusion device 10. As shown more clearly in FIG. 3C, the outer catheter 20 houses an inner catheter 40. The capsule 30′ is located at a distal end 20b, 40b of the outer catheter 20 and the inner catheter 40. The capsule 30′ may be securely connected to the distal ends 20b, 40b of the outer and inner catheters 20, 40 or may be formed as part of the tubular body 22, 42 of the outer catheter 20 or the inner catheter 40.

The outer catheter 20 has an clongate tubular body 22 and a central lumen 24 (shown more clearly in FIG. 3B) that extends from a proximal end 20a of the catheter 20 to the distal end 20b of the catheter. The catheter 20 extends to a distal aperture 23 with a surrounding distal face 25. As shown schematically in FIG. 2, the proximal end 20a of the outer catheter 20 is housed in a handle 200. The proximal end 20a of the catheter therefore remains outside of the patient's body while the distal portion 20b is advanced through the patients' vasculature 6 towards the left atrial appendage 50. A medical practitioner manipulates the position and orientation of the outer catheter 20 by using the handle 200. As shown, the handle 200 may also be connected to any number of systems known to those skilled in the art, generally indicated by system 250 in FIG. 2.

The distal end 20b of the outer catheter 20 includes a plurality of apertures 26a-e. The apertures 26a-e are in fluid communication with the central lumen 24 of the outer catheter 20. In this example, four apertures 26a-d are positioned radially around the distal face 25 of the catheter 20. In this example, the apertures 26a-d are in fluid communication with the central lumen 24 of the outer catheter 20. As will be discussed further below, the apertures 26a-d arc arranged to deliver fluid 70 into an internal volume 13 of the occlusion device 10 to radially expand the occlusion device 10.

A fifth aperture 26e is positioned on the tubular body 22 of the outer catheter 20. In this example, the fifth aperture 26e extends perpendicularly from the central lumen 24. However, the fifth aperture 26e may be positioned at any position along the length of the tubular body 22 of the catheter 20. As will be discussed further below, the fifth aperture 26e is arranged to deliver contrast 75a after the occlusion device 10 has been securely positioned into the left atrial appendage 50. In this example, the outer catheter 20 includes four exemplary apertures 26a-d and one fifth aperture 26e. However, it will be understood that any suitable number or orientation of apertures 26a-e may be used to deliver fluid 70 and/or contrast 75a through the outer catheter 20 to the distal end 20b of the outer catheter 20.

An inner catheter 40 is housed inside the central lumen 24 of the outer catheter 20. The outer and inner catheters 20, 40 form a nested arrangement. The inner catheter 40 has an clongate tubular body 42 and a central lumen 44 that extends from a proximal end 40a to a distal end 40b of the inner catheter 40. A plurality of locking wires 47 (shown in more detail in FIG. 3C) are housed in the central lumen 44 of the inner catheter 40. The locking wires 47 are selectively deployable by the medical practitioner to secure the inner catheter 40 to an outer surface 19 of the occlusion device 10 (discussed further below). The inner catheter 40 extends to an enlarged tip 43, which in this example, is substantially cylindrical with a curved wall 43a and an end face 43b. As shown, the enlarged tip 43 extends over the central lumen 44 of the inner catheter 40.

The enlarged tip 43 includes a plurality of apertures 46 that are arranged radially around the curved wall 43a. Although only two exemplary apertures 46 are shown in FIG. 3B, it will be understood that any number and/or orientation of apertures 46 may be positioned on the enlarged tip 43. In a deployed state, the locking wires 47 are arranged to extend through the apertures 46. Preferably, the apertures 46 extend around the curved wall 43a of the enlarged tip 43. As shown more clearly in FIG. 3C, when the wires 47 are in a deployed position, the wires extend through the apertures 46 are engage with an outer surface 19 of the occlusion device 10. When the wires 47 are in a retracted position (as shown in FIG. 3B), the wires 47 are fully housed within the central lumen 44 of the inner catheter 40.

A proximal portion 14 of the occlusion device 10 is secured to the distal end 20b of the outer catheter 20, and a distal portion 18 of the occlusion device 10 is secured to the distal end 40b of the inner catheter 40. The occlusion device 10 is therefore housed inside the central lumen 24 of the outer catheter 20 in a collapsed state 10a.

As shown in FIG. 3C, the capsule 30′ extends over the distal end 20b of the outer catheter 20, the distal end 40b of the inner catheter 40 the occlusion device 10. The capsule 30′ holds the occlusion device 10 in a collapsed state 10a. The capsule 30′ protects the occlusion device 10 as the distal end 20b of the outer catheter 20 navigates through the vasculature 6 of the patient. The capsule 30′ is arranged to be retracted proximally by a medical practitioner (i.e. towards the proximal end 20a of the outer catheter 20) to selectively deploy the occlusion device 10 from a collapsed state 10a to an expanded state 10b. The occlusion device 10 is therefore unsheathed by the capsule 30′. As the capsule 30′ is retracted proximally, the occlusion device 10 automatically expands into a partially expanded state 10b′. The occlusion device 10 is arranged to occlude (or obstruct) a left atrial appendage 50.

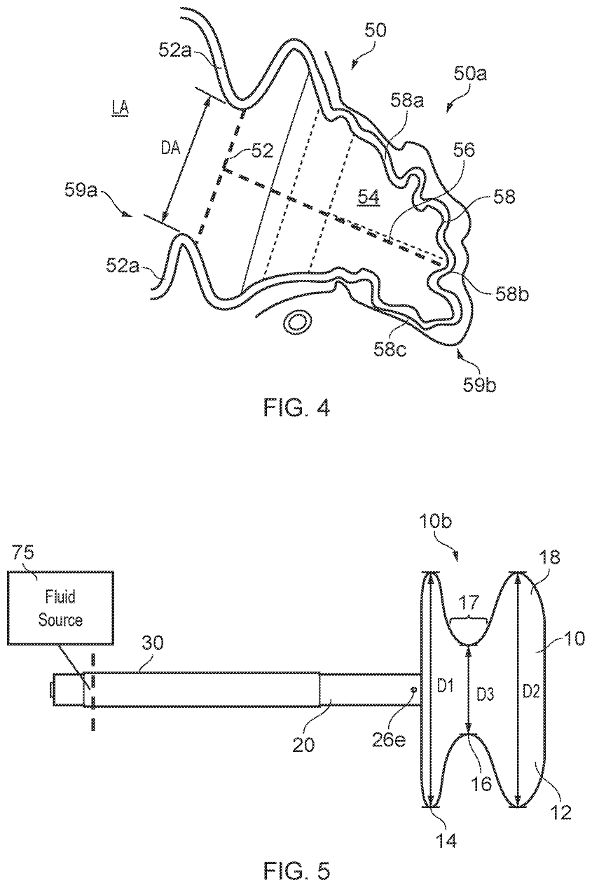

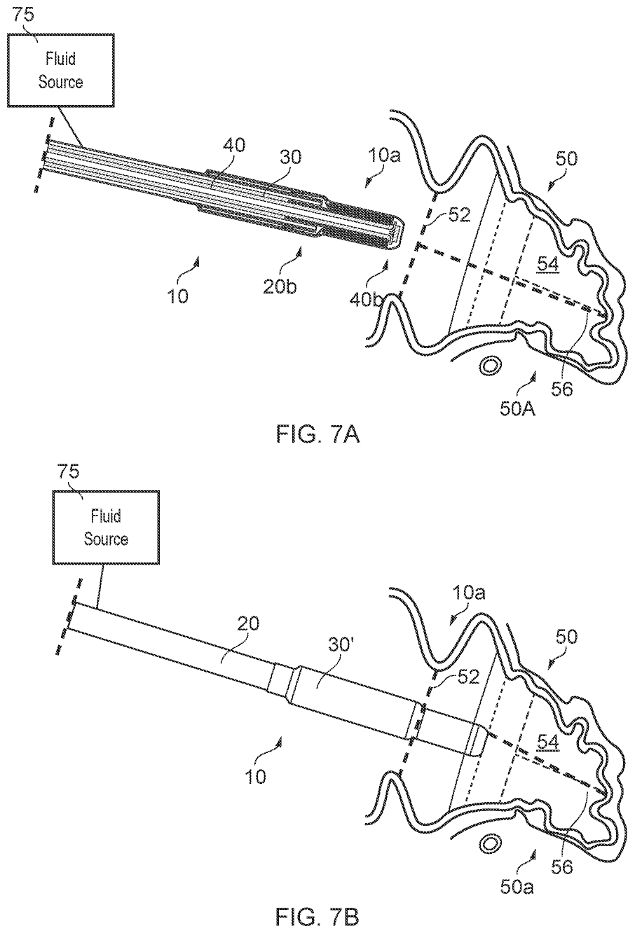

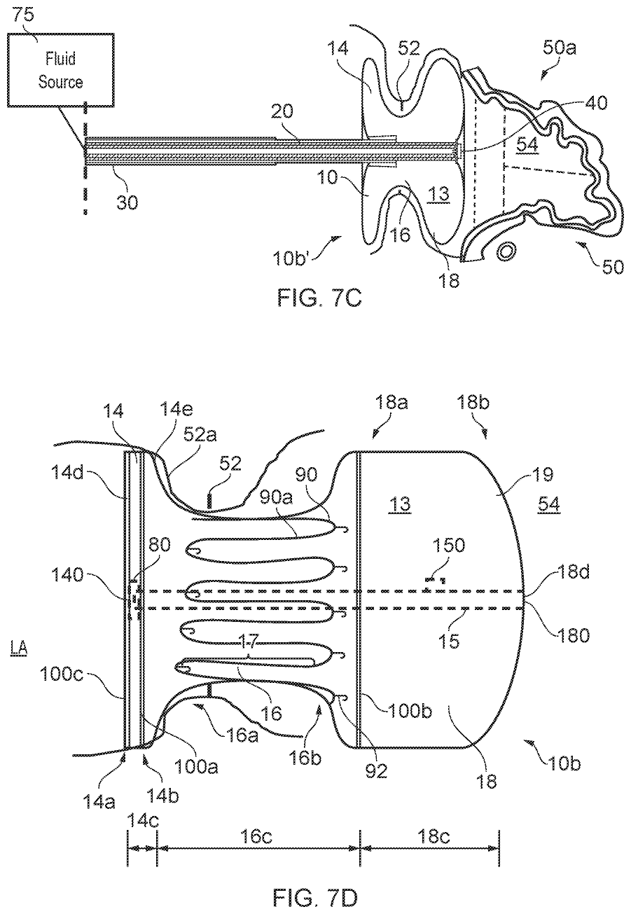

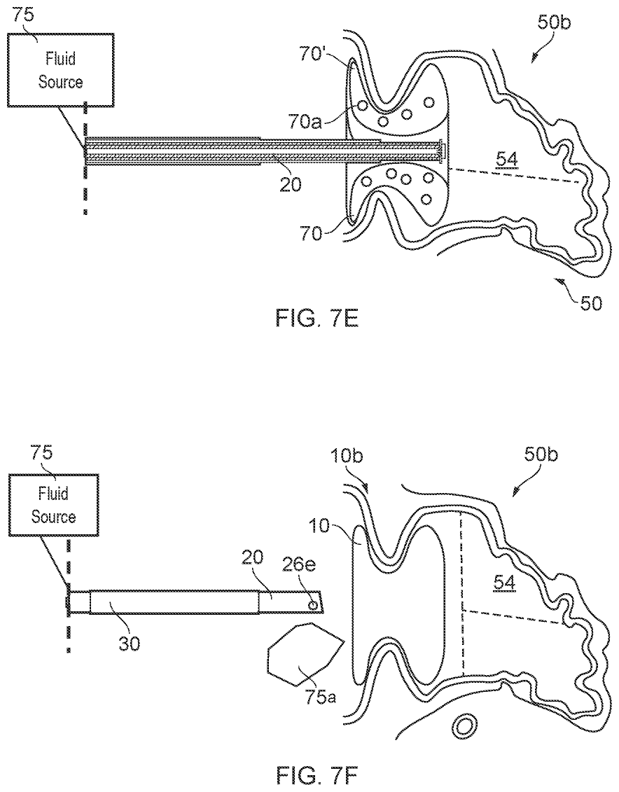

As shown in FIG. 7A, the distal end 20b, 40b of the inner and outer catheters 20, 40 are arranged to be positioned next to the left atrial appendage 50 within the heart 45. While the shape of left atrial appendage 50 will vary for among patients, the left atrial appendage 50 is shown in FIG. 4 as being generally bursiform (pouch) shaped and has an opening 52 at a proximal end 59a that extends to a distal end 59b. The opening 52 is generally circular in shape and has a diameter DA.

The left atrial appendage 50 has an internal volume 54 that is in fluid communication with the left atrium LA. The internal volume 54 may contain a volume of blood from the heart 45. The internal volume 54 is partially enclosed by a wall 58. As shown, the wall 58 has an irregular shape that generally forms an car-shaped structure. In this example, the left atrial appendage wall 58 is formed of a superior wall 58a, an inferior wall 58c and a rear wall 58b. The left atrial appendage wall 58 includes further anterior and posterior wall sections that are not shown in FIG. 4.

The left atrial appendage 50 may have any type of morphology. For example, the left atrial appendage 50 may have a depth greater than the opening diameter DA, a smaller than the opening diameter DA, have a chicken-wing shape, cactus shape or cauliflower shape.

The left atrial appendage has a depth 56 that extends from the opening 52 (at the proximal end 59a) to the rear wall 58b (at the distal end 59b). Generally, the depth 56 of the left atrial appendage 50 is greatest at the rear wall 58b. The left atrial appendage 50 is in a natural, pre-treated state 50a in FIG. 4. As shown, the width of the left atrial appendage 50 varies along a depth 56 of the left atrial appendage 50. In this example, the diameter DA of the opening 52 does not generally correspond to the widest part of the left atrial appendage 50.

An exemplary occlusion device 10 for occluding a left atrial appendage 50 is shown in FIG. 5. As shown, the occlusion device 10 is in an expanded state 10b with the capsule 30′ in a proximally retracted position. The occlusion device 10 is arranged to move between a collapsed position 10a, a partially expanded position 10b′ and a fully expanded position 10b. The distal ends 20b, 40b of the inner and outer catheters 20, 40 are arranged to be removably attached to the occlusion device 10 by the medical practitioner after the occlusion device 10 has been securely positioned in the left atrial appendage 50. The occlusion device 10 is therefore arranged to remain in the left atrial appendage 50 for an extended period of time.

The occlusion device 10 has an expandable hollow body 12. As shown more clearly in FIG. 7D, the body 12 of the occlusion device 10 has an internal volume 13. The occlusion device 10 includes a proximal portion 14 with a first diameter D1. The proximal portion 14 extends from a proximal end 14a to a distal end 14b. As discussed below, the proximal portion 14 is arranged to sealingly engage against the opening 52 of the left atrial appendage 50. The proximal portion 14 is arranged proximal to the distal ends 20b, 40b of the inner and outer catheters 20, 40. The outer catheter 20 is releasably connected to the proximal portion 14 through a closure device 80, which is discussed further below. The occlusion device 10 also includes a distal portion 18 with a second diameter D2. The distal portion 18 extends from a proximal end 18a to a distal end 18b. The distal portion 18 is arranged to be positioned inside and at least partially fill the left atrial appendage 50. The distal portion 18 of the occlusion device is releasably connected to the inner catheter 40 through the locking wires 47. In this example, the first diameter D1 of the proximal portion 12 and the second diameter D2 of the distal portion 18 are substantially similar. However, the first diameter D1 and the second diameter D2 may be any size, for example, D1 may be larger than D2. Preferably, D1 and D2 are substantially similar in size so that the proximal portion 14 effectively seals opening 52 of the left atrial appendage 50 and the distal portion 18 occupies a relatively large volume of the internal volume 54 of the left atrial appendage.

The occlusion device also includes central portion 16 that extends between the proximal 14 and the distal portions 18. The central portion extends between a proximal end 16a to a distal end 16b. The proximal end 16a of the central portion 16 is connected to the distal end 14b of the proximal end 14. Similarly, the distal end 16b of the central portion 16 is connected to the proximal end 18a of the distal end 18. The central portion 16 includes a waist region 17 with a third diameter D3. As shown, the third diameter D3 is smaller than the first and second diameters D1, D2 of the proximal and distal portions 14, 18. The occlusion device 10 is arranged to move from a collapsed state 10a (shown in FIGS. 3C, 7A and 7B) to an expanded state 10b (shown in FIG. 7D-7F). The occlusion device 10 is in a collapsed state 10a (i.e. not expanded) when the occlusion device 10 is housed inside the capsule 30′. The occlusion device 10 is therefore in a collapsed state 10a as the catheters 20, 40 are moved through the patient's vasculature 6 towards the left atrial appendage 50.

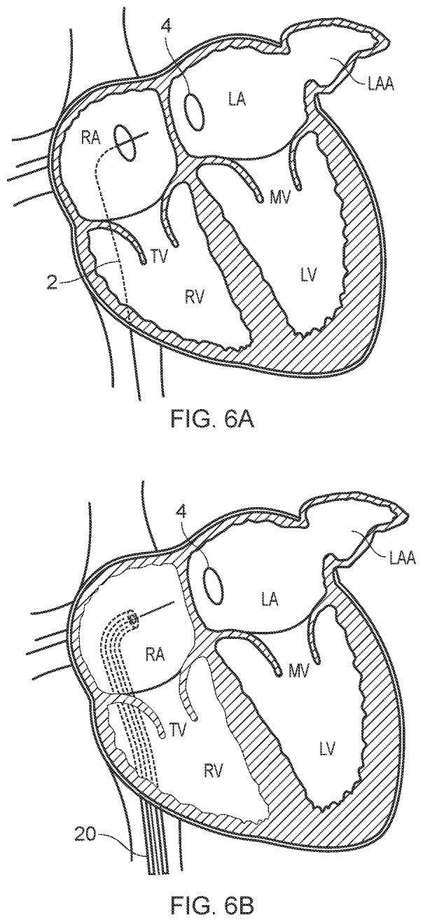

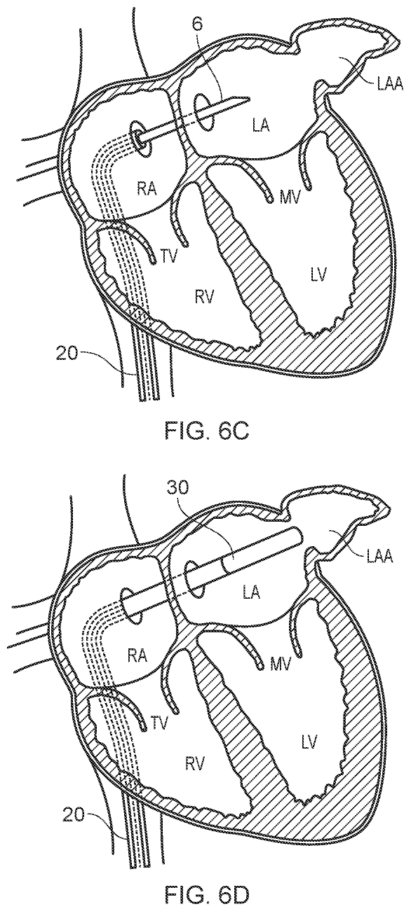

Referring to FIGS. 6A to 6D, a method for delivering and deploying the occlusion device 10 to a left atrial appendage LAA will now be described. In an exemplary embodiment, a guidewire 2 is advanced after having been introduced into the vasculature via a percutaneous entry point and tracked through the vasculature into a left atrium LA of a heart. Intravascular access to the right atrium RA may be achieved via a percutaneous access site to femoral venous access up to the inferior venal cava, or other known access routes. Thereafter, a guidewire is advanced through the circulatory system, eventually arriving at the heart.

The guidewire 2 is directed into the right atrium RA (see FIG. 6A), traverses the right atrium and is made to traverse, with the aid of a pre-existing hole 4 (see FIG. 6B) or a transseptal needle 6 (see FIG. 6C) an atrial septum, thereby entering the left atrium LA. Once the guidewire 2 is positioned, the entry port and the atrial septum are dilated to permit entry of the catheter 20 into the left atrium LA towards the left atrial appendage LAA (see FIG. 6D).

There, the capsule 30′ of the catheter 20 is positioned proximate the left atrial appendage LAA. Although described as a transfemoral antegrade approach for percutancously accessing left atrium LA, the catheter 20 may be positioned within the desired arca of the heart via different methods or routes. For example, and not by way of limitation, another possible path would be through the radial vein into the brachial vein, through the subclavian vein, through the superior vena cava into the right atrium, and then transeptally into the left atrium.

Yet another possible path would be through the femoral artery into the aorta (as shown in FIG. 2), through the aortic valve into the left ventricle, and then retrograde through the mitral valve into the left atrium. In another embodiment, the left ventricle LV may be accessed via a transapical approach, and the catheter 20 may be advanced through the left ventricle LV, the mitral valve, and into the left atrium LA adjacent the left atrial appendage LAA. In addition, although described with the use of a guidewire, in another embodiment hereof the catheter 20 may access the left atrium LA without the use of a guidewire.

As shown in FIG. 7A, which shows a cross-sectional view of the capsule 30′ in a deployed state, the occlusion device 10 remains in a collapsed state 10a when the distal end 20b, 40b of the catheters 20, 40 are positioned near the opening 52 of the left atrial appendage 50. The distal end 20b, 40b of the catheters 20, 40 are advanced through the opening 52 and into the volume 54 of the left atrial appendage 50, as shown in FIG. 7B. In an alternative arrangement, the capsule 30′ remain outside the opening 52 of the left atrial appendage 50 before the capsule 30′ is proximally retracted.

When the capsule 30′ is proximally retracted in FIG. 7C, the occlusion device 10 automatically expands into a partially expanded state 10b′. The occlusion device 10 may include an expansion mechanism, such as reinforcement struts 90 (discussed further below), which helps the occlusion device 10 to automatically partially expand into a partially expanded state 10b′. In a partially expanded state 10b′, the first, second and third diameters D1-D3 of the occlusion device 10 are not at a maximum length, or the internal volume 13 of the occlusion device does not contain any fluid 70. The occlusion device 10 is arranged to receive a fluid 70 into the internal volume 13 of the hollow body 12 so that the occlusion device 10 becomes fully expanded, and is in a fully expanded state 10b. In a fully expanded state 10b, the first, second and third diameters D1-D3 of the occlusion device 10 are at a maximum length as permitted by the surrounding patient anatomy (i.e. the walls 58a-c of the left atrial appendage 50) or the internal volume 13 of the occlusion device 10 contains fluid 70.

The capsule 30′ may be retracted after the distal ends 20b, 40b of the catheters 20, 40 are advanced through the opening 52 of the left atrial appendage 50 to ensure that at least the distal portion 18 of the occlusion device 10 is positioned inside the volume 54 of the left atrial appendagc 50. In an alternative arrangement, the capsule 30′ may be proximally retracted before the distal ends 20b, 40b of the catheters 20, 40 are advanced through the opening of the atrial appendage 50. In this instance, the catheters 20, 40 are used to distally advance the occlusion device 10 into the correct position, i.e. so that the distal portion 18 of the occlusion device 10 is positioned inside the internal volume 54 of the left atrial appendage 50, and the central portion 16 is aligned with the opening 52 of the left atrial appendage 50.

FIG. 7D shows a schematic drawing of another exemplary occlusion device 10 in a fully expanded state 10b. As shown, the occlusion device 10 includes a central lumen 15 that extends from a proximal opening 140 at the proximal end 14a of the proximal portion 14 to a distal opening 180 at the distal end 18b of the distal portion 18. The central lumen 15 includes at least one aperture 150 (shown schematically in FIG. 7D) that extends from the central lumen 15. The aperture 150 is in fluid communication with the internal volume 13 of the expandable hollow body 12 of the occlusion device 10. Fluid 70 is arranged to be delivered to the internal volume 13 of the occlusion device 10 (as shown in FIG. 7E) with a fluid source 75 that is in fluid communication with the outer catheter 20. The occlusion device 10 is arranged to move between a partially expanded state 10b′ and an expanded state 10b after fluid 70 (in this example, biogel 70′) is introduced into the internal volume 13 of the hollow body 12 with the fluid source 75.

As shown more clearly in FIG. 3B and 7D, the distal end 20b of the outer catheter 20 is partially housed in the central lumen 15. The inner catheter 40 extends towards the distal opening 180 and is housed along the entire length of the central lumen 15. As shown, the distal end 40b of the inner catheter 40 extends beyond the distal opening 180.

The distal ends 20b, 40b of the catheters 20, 40 may be secured to the occlusion device 10 when the occlusion device 10 is loaded into the capsule 30′. Alternatively, the occlusion device 10 may not be pre-mounted onto the distal ends 20b, 40b of the catheters 20, 40. After the capsule 30′ has been proximally retracted, the medical practitioner may distally advance and position the occlusion device 10 by inserting the distal ends 20b, 40b of the catheters 20, 40 through the central lumen 15 of the occlusion device 10.

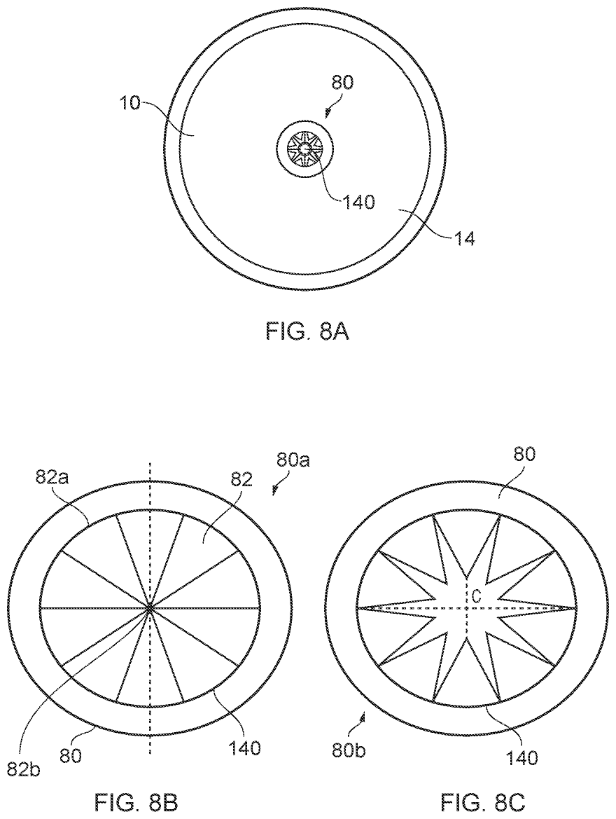

The occlusion device 10 includes a closure 80 at the proximal portion 14 of the occlusion device 10 (shown more clearly in FIGS. 8A-8C). The closure 80 is arranged around the proximal opening 120 of the central lumen 15. The closure 80 is moveable between a first position 80a and a second position 80b. In the first position 80a, the closure 80 obstructs the proximal opening 140 and prevents the flow of fluid 70 in and out of the central lumen 15 through the proximal opening 140. In the second position 80b, the closure 80 permits the flow of fluid 70 in and out of the central lumen 15 through the proximal opening 140.

In this example, the closure 80 is formed from a plurality of leaflets 82. As shown in more detail in FIG. 8B, the leaflets 82 are arranged in a radial configuration. Each leaflet 82 is secured at a fixed end 82a and extend to a free end 82b. The free end 82b is positioned in a centre C of the proximal opening 140. In a first position 80a, the leaflets 82 extend over the proximal opening 140. As the capsule 30′ is retracted proximally and the occlusion device 10 begins to expand, the distal ends 20b, 40b of the catheters 20, 40 extend through the central lumen 15 of the occlusion device 10 and push against the leaflets 82. The leaflets 82 are therefore arranged to move between the first position 80a and the second position 80b by being pushed inwardly by the distal ends 20b,40b of the catheters 20,40.

Once the catheters 20, 40 are detached from the occlusion device 10 and are proximally withdrawn, the leaflets 82 are arranged to return to the first position 80a. Therefore, the leaflets 82 act as a one-way valve closure that allows the distal end 20b, 40b of the catheters 20, 40 to enter the central lumen 15, but prevent any fluid 70 inside the occlusion device 10 from escaping through the proximal opening 140 and into the left atrium LA.

Preferably, the leaflets 82 are at least partially formed from nitinol so that the leaflets 82 are pliable to move between the first and second positions 80a, 80b. Forming the leaflets 82 from nitinol also enable the leaflets 82 to automatically return to a first position 80a as the catheters 20, 40 are withdrawn from the central lumen 15. Forming the leaflets 82 from nitinol is also advantageous because the rigidity of nitinol changes depending on the temperature of the leaflets 82. For example, the leaflets 82 remain pliable as the occlusion device 10 is first positioned in the left atrial appendage because the occlusion device 10 is typically stored in chilled saline prior to implantation. Once the occlusion device 10 is held in an expanded state 10b and left in the left atrial appendage 50, the surrounding body temperature causes the leaflets to become rigid, which helps prevent fluid 70 from flowing back out through the closure 80.

In this example, the leaflets 82 are shown to be substantially triangular shaped and arranged in a radial configuration. However, the leaflets 82 may be any suitable shape or configuration and may be mechanically actuated between the first and second positions 80a, 80b. For example, the leaflets 82 may be semi-circular shaped.

As shown in FIG. 7C, the distal end 20b of the outer catheter 20 extends beyond the closure 80 when the occlusion device 10 is in a partially expanded state 10b′. The distal end 20b of the outer catheter 20 is not mechanically secured to the central lumen 15 of the occlusion device 10 and may be proximally retracted from the central lumen 15 or distally advanced by the medical practitioner as required. The distal end 40b of the inner catheter 40 is releasably secured to the distal portion 18 of the occlusion device 10.

The distal end 40b of the inner catheter 40 is advanced through the central lumen 15 until the distal end 40b of the inner catheter 40 extends beyond the distal aperture 180. Then, the medical practitioner may deploy the locking wires 47 through the apertures 46 on the enlarged tip 43 so that they engage with the outer surface 19 of the occlusion device 10 at the distal end 40. This helps position and secure the inner catheter 40 in the occlusion device 10 as the occlusion device is expanded into an expanded state 10b. The retractable wires 47 may be disengaged from the occlusion device 10 (i.e. so that they no longer extend through the apertures 46 on the inner catheter 40 and remain inside the central lumen 44 of the catheter 40) by the medical practitioner.

The locking wires 47 may be deployed as fluid 70 is being delivered to the occlusion device 10 to help secure the occlusion device 10 in position. The locking wires 47 may be retracted to disconnect the inner catheter 40 from the occlusion device 10. The inner catheter 40 may then be proximally retracted through the central lumen 15 of the occlusion device 10 and into the central lumen 24 of the outer catheter 20, before both catheters 20, 40 are proximally retracted away and withdrawn from the occlusion device.

The central lumen 15 provides fluid communication between the central lumen 24 of the outer catheter 20 (through the apertures 26a-d) and internal volume 13 of the occlusion device 10 (through the aperture 150). In this example, only one aperture 150 extends from the central lumen 15 of the occlusion device 10 to the internal volume 13. However, any number of apertures 150 may be arranged along the length of the central lumen 15. The central lumen 15 also provides fluid communication between the central lumen 24 of the outer catheter 20 and the internal volume 54 of the left atrial appendage 50. As shown more clearly in FIG. 7D, the distal opening 180 (at the distal end of the central lumen 15) is in fluid communication with the internal volume 54 of the left atrial appendage 50, as shown more clearly in FIG. 7D.

The expandable body 12 of the occlusion device 10 is arranged to move between a collapsed state 10a (shown in FIGS. 7A and 7B), a partially expanded state 10b′, an expanded state 10b (shown in FIGS. 7C and 7D). As described earlier, the expandable body 12 at least partially expands through the introduction of a fluid 70 into the internal volume 13. The fluid 70 is introduced by the fluid source 75 that is in fluid communication with the outer catheter 20. The fluid 70 is arranged to pass through the central lumen 24 of the outer catheter 20, through the apertures 26a-d on the distal face 25 of the catheter 20, into the central lumen 15 of the occlusion device 10 and through the aperture 150 into the internal volume 13. The occlusion device 10 is arranged to radially expand as the fluid 70 enters into the internal volume 13.

In an expanded state 10b, the distal portion 18 of the occlusion device 10 is arranged to occlude the internal volume 54 of the left atrial appendage. As shown in FIGS. 4 and 7C, the distal portion 18 is distal to the proximal portion 14 of the occlusion device 10 and is arranged to be positioned inside the internal volume 54 of the left atrial appendage 50. The distal portion 18 has a width (i.e. the diameter D2) and a depth 18c. The second diameter D2 of the distal portion 18 is arranged to extend across the width of the left atrial appendage 50 (i.e. from walls 58a to 58c). The depth 18c of the distal portion 18 extends between the proximal end 18a and the distal end 18b of the distal portion. The depth 18c of the distal portion 18 is arranged to extend along the depth 56 of the left atrial appendage 50 (i.e. from the opening 52 to the rear wall 58b).

As shown, the distal end 18b of the distal portion 18 is substantially domed and has an apex 18d that in this example, generally corresponds to the distal opening 180. The dome shape of the distal portion 18 enables the occlusion device 10 to occupy a large volume of the left atrial appendage 10 in an expanded state 10b. The distal portion 18 may be any suitable size, but preferably, the distal portion 18 is sized to match the internal volume 54 of the left atrial appendage 50. Preferably, the diameter D3 of the distal portion 18 extends substantially across the width of the left atrial appendage 50. The third diameter D3 of the distal portion 18 may be substantially oval or elliptical shaped so that the distal portion 18 generally conforms to the internal profile of the left atrial appendage 50.

In an expanded state 10b, the proximal portion 14 is arranged to sealingly engage against the opening 52 of the left atrial appendage 50, as shown in FIG. 7D. The proximal portion 14 has a width (i.e. the diameter D1) and a depth 14c. The first diameter DI of the proximal portion 14 is arranged to extend across the width of the left atrial appendage opening 52 so that the proximal portion 14 obstructs the entire opening 52. The depth 14c of the proximal portion 14 extends between the proximal end 14a and the distal end 14b of the proximal portion.

The proximal portion 14 has a proximal face 14d at the proximal end 14a, and a distal face 14c at the distal end 14b. In this example, the proximal face 14d of the proximal portion 14 is substantially planar, while the distal face 14e generally conforms to the curved profile of the area 52a that surrounds opening 52 of the left atrial appendage 50. In an expanded state 10b, the distal face 14e of the proximal portion 14 engages the left atrial appendage opening 52 by contacting against the area 52a that surrounds the opening 52 of the left atrial appendage 50. By contacting against the arca 52a, the proximal portion 14 obstructs the opening 52 so that the left atrial appendage 50 is no longer in fluid communication with the left atrium LA.

In this example, the proximal face 14d of the proximal portion 14 is substantially planar. The proximal face 14d of the proximal portion 14 therefore does not protrude excessively into the left atrium LA of the heart 45. This prevents obstructions of flow in the left atrium LA, minimising the risk of adverse flow patterns in the left atrium LA.

The proximal portion 14 and the distal portion 18 of the occlusion device 10 are connected via a central portion 16. The central portion 16 is substantially cylindrical in shape and has a depth 16c that extends from a proximal end 16a to a distal end 16b. The central portion 16 also includes a waist region 17. As shown, the diameter D3 of the waist region 17 is smaller than the diameter of the proximal end 16a (which corresponds to the first diameter D1 of the proximal end 14) and is smaller than the diameter of the distal end 16b (which corresponds to the second diameter D3 of the distal end 18). Consequently, the third diameter D3 of the waist region 17 is smaller than the first diameter D1 of the proximal portion 14 and smaller than the third diameter D3 of the distal portion 18. The central portion 16 of the occlusion device 10 is arranged to extend across the opening 52 of the left atrial appendage 50.

The central portion 16 of the occlusion device 10 is formed from a woven material. Preferably, the waist region 17 of the central portion 16 is formed of a woven material that is capable of stretching up to twice its unloaded length, such as polyetheretherketone (PEEK). The woven material may be tested under ASTM test D5034. Forming the central region 16 from a woven material enables occlusion device 10 to be secured in the left atrial appendage 50 when the occlusion device 10 is in an expanded state 10b.

In an expanded state 10b, the internal volume 13 of the occlusion device 10 is filled with fluid 70. This at least radially expands the central portion 16 so that the waist region 17 of the central portion 16 abuts against the tissue inside the opening 52 of the left atrial appendage. The central portion 16 therefore exerts a radial force against the tissue inside the opening 52. In an expanded state 10b, the central portion 16 forms an interference fit between the opening 52 and the waist region 17 of the occlusion device 10. This secures the occlusion device 10 in the opening 52 of the left atrial appendage. As the central portion 16 is formed from woven material, the central portion 16 can easily deform and conform to the morphology of the opening 52. By using a combination of fluid 70 and woven fabric, it is easier for the central portion 16 to radially expand and conform to the anatomy of the opening 52 to provide a secure fit. In an expanded state 10b, the third diameter D3 of the waist region 17 generally conforms to the diameter DA of the left atrial appendage opening 52. Therefore, the occlusion device 10 can be used on any number of patients with different morphologies.

As described earlier, the occlusion device 10 is arranged to be implanted into the left atrial appendage 50 for extended periods of time. The central portion 16 may include a plurality of reinforcement struts 90 that are arranged to strengthen the central portion 16. The reinforcement struts 90 are arranged to strengthen the central portion 16 radially, so that the waist region 17 can effectively abut against the opening 52 of the left atrial appendage 50 in an expanded state 10b. The reinforcement struts 90 therefore help secure the occlusion device 10 to the left atrial appendage opening 52, and help prevent the occlusion device 10 from becoming dislodged and migrating into the left atrium LA. The reinforcement struts 90 also help prevent the central portion 16 from collapsing inwardly (i.e. towards the central lumen 15).

In this example, the reinforcement struts 90 are formed from a nitinol wire 90a that extends across the majority of the depth 16c of the central portion 16. As shown in FIG. 7D, the nitinol wire 90a is one continuous wire that extends in a serpentine pattern around a circumference of the central region 16. However, in other examples, the reinforcement struts 90 may include any number of struts that extend around the central region 16. When the capsule 30′ is proximally retracted, the reinforcement struts 90 may also help to automatically partially expand the occlusion device 10 into the partially expanded state 10b′.

In this example, the reinforcement strut 90 is substantially aligned with the depth 16c of the central portion 16 (i.e. extends from the proximal end 16a to the distal end 16b of the central portion). In other examples, the reinforcement struts 90 may extend radially around the central portion 16 (i.e. parallel with the waist region 17).

The reinforcement struts 90 may be inside the woven material that forms the central portion 16. In this example, the reinforcement struts 90 do not contact tissue forming the opening 52a of the left atrial appendage 50. In another example, the reinforcement struts 90 may be positioned on the outer surface 19 of the central portion 16. In this instance, the reinforcement struts 90 may be secured to the outer surface 19 of the central portion through any suitable means, such as suturing, using adhesive or bonding the reinforcement struts 90 to the outer surface 19 of the central portion.

In yet another example, a portion of the reinforcement strut 90 may protrude from the woven material that forms the central portion 16. For example, the reinforcement struts 90 may include at least one barb 92, which, as shown in FIG. 7D, is a generally pronged protrusion that extends from the nitinol wire 90a. The barb 92 may be arranged to protrude through the outer surface 19 of the occlusion device 10 and engage with the left atrial appendage 50 tissue, or more specifically, the tissue surrounding forming the opening 52 of the left atrial appendage 50. The barbs 92 mechanically secure central portion 16 to the opening 52 of the left atrial appendage 50 to help prevent the occlusion device 10 from migrating into the left atrium LA. It will be understood that any reinforcement strut 90 arrangement may be used, and there may be any number or arrangement of barbs 92 from the reinforcement struts 90.

The proximal portion 14 and the distal portion 18 of the occlusion device 10 may also be formed from a woven material. The woven material that forms the distal portion 18 may have different characteristics than the woven material that forms the proximal portion 14, or alternatively, the woven material that forms the distal portion 18 may be identical to the woven material that forms the proximal portion 14.

Preferably, the proximal portion 14 and the distal portion 18 are formed from the same woven material. The woven material that forms the distal portion 18 and the proximal portion 14 has different characteristics than the woven material that forms the central portion 16. In this example, the woven material that forms the proximal portion 14 and the distal portion 18 is less compliant than the woven material that forms the waist region 17. For example, the woven material that forms the proximal and distal portions 14, 18 may stretch less than approximately 20% of its unloaded length and may be, for example, polyester (PET). By forming the proximal portion 14 and the distal portion 18 from a woven material that is stiffer than the central portion 16, the proximal and distal portions 14, 18 creates a “corking effect” which helps prevent the occlusion device 10 from migrating back into the left atrium LA.

The woven material that forms the central portion 16 and the woven material that forms the proximal and distal portions 14, 18 may be any suitable biocompatible material that possesses suitable pore size to promote cellular integration of the occlusion device 10 into the surrounding tissuc. Additionally, the pore size of the woven material(s) is preferably small enough to prevent leakage of fluid 70 from the internal volume 13 of the occlusion device 10. For example, the pore size of the woven material may be approximately 40-100 microns. The woven material(s) preferably have a thickness of 0.15-0.5 mm.

Forming the occlusion device 10 from woven materials is advantageous as the woven material naturally acts as a cellular scaffold and thereby promotes tissue healing and integration of the occlusion device 10 in areas that the occlusion device 10 contacts the left atrial appendage (such as the waist region 17, or the distal face 14e of the proximal portion 14).

As described carlier, the fluid source 75 is operable to deliver fluid 70 through the outer catheter 20 into the internal volume 13 of occlusion device 10. The expandable hollow body 12 of the occlusion device 10 is arranged to expand as the fluid 70 enters into the internal volume 13 of the body 12. The fluid 70 is preferably a biogel 70′, such as. . . . However, any suitable fluid may be used, preferably with sufficient viscosity and surface tension to prevent leakage through the woven material. The fluid 70 is introduced into the internal volume 13 and the medical practitioner may control how much fluid 70 is introduced to expand the distal 18, central 16 and proximal portion 14 of the occlusion device 10. By using a combination of fluid 70 and woven fabric, it is casier for the occlusion device 10 to expand and conform to the anatomy of the left atrial appendage 50 to ensure that the occlusion device is securely seated in the opening 52 of the left atrial appendage 50.

The fluid 70 may include radiopaque markers 70a so that the injection and delivery of fluid 70 into the occlusion device 10 may be observed by the medical practitioner through additional imaging techniques, such as fluoroscopy. The rate of expansion of the occlusion device 10 may therefore be monitored in real-time by the medical practitioner while the occlusion device 10 is located in situ (i.e. in the left atrial appendage 50). This assists the medical practitioner in ensuring that the occlusion device 10 has expanded enough to occupy the internal volume 54 of the left atrial appendage, and that the central portion 16 is securely seated in the opening 52 left atrial appendage 50. The amount of fluid 70 required for the occlusion device 10 to be in an expanded state 10b may vary depending on the size of the left atrial appendage 50, which can vary depending on the morphology of the patient. The radiopaque markers 70a therefore also help guide the medical practitioner to provide the correct amount of fluid 70 into the hollow body 12 to move the occlusion device 10 from a partially expanded state 10b′ to an expanded state 10b.

The occlusion device 10 may include a number of circumferential bands 100a-c that extend at least partially around the circumference of the body 12 of the occlusion member 10. In this example, the occlusion device 10 has three exemplary circumferential bands 100a-c. The circumferential bands 100a-c are formed on the woven material and preferably, at least partially comprise radiopaque material. The circumferential bands 100a-c may therefore be observed by the medical practitioner when the occlusion device 10 is positioned near or in the left atrial appendage 50. Preferably, the radiopaque material that forms the circumferential bands 100a-c include radiopaque ink. The radiopaque ink can be easily and accurately positioned on the woven material that forms the expandable body 12 of the occlusion device 10 before the occlusion device 10 is collapsed and positioned inside the capsule 30′.

As will be described further below, the circumferential bands 100a-c act as “fill lines” which delimit the different portions of the occlusion device 10 (e.g. the distal portion 18, the central portion 16 and/or the proximal portion 14). The medical practitioner may therefore use the circumferential bands 100a-c to help identify that the occlusion device 10 has been correctly positioned and determine when the occlusion device 10 has been filled with an adequate amount of fluid 70.

In this example, the occlusion device 10 includes a first circumferential band 100a that extends at least partially around the proximal end 16a of the central portion 16, and a second circumferential band 100b that extends at least partially around the distal end 16b of the central portion 16. The first and second circumferential bands 100a, 100b are substantially symmetrical. This allows the medical practitioner to observe both circumferential bands 100a, 100b without rotating the occlusion device 10. The medical practitioner can visually observe how much fluid 70 is inserted between both circumferential bands 100a, 100b to establish whether or not the occlusion device 10 is positioned correctly and has enough fluid 70 to form an interference fit between the waist region 17 of the occlusion device 10 and the opening 52 of the left atrial appendage 50.

The occlusion device 10 may also include a third circumferential band 100c that extends at least partially around the proximal portion 14 of the occlusion device. In this example, the third circumferential band 100c extends around the proximal face 14d of the proximal portion. The medical practitioner may visually observe how much fluid 70 has inserted adjacent to the the third circumferential band 100c to establish whether or not the internal volume 13 of the occlusion device 10 has been fully filled with fluid 70. For example, the medical practitioner may insert fluid 70 into the occlusion device 10 until inserted fluid 70 extends along the entire length of the circumferential band 100c.

The occlusion device 10 in an expanded state 10b is shown in FIGS. 7D and 7E. As shown, the internal volume 13 of the occlusion device is filled with fluid 70. As the occlusion device 10 expands, the opening 52 of the left atrial appendage 50 is sealed with the proximal portion 14 of the occlusion device 10. The internal volume 54 of the left atrial appendage 50 is occluded with the distal end 18 of the occlusion device 10. The occlusion device 10 is arranged to be implanted in the left atrial appendage 50 for an extended period of time.

The catheters 20, 40 are releasably connected to the occlusion device 10 and may be detached by the medical practitioner by. For example, a portion of the central lumen 15 that is near the proximal opening 140 of the device may be reinforced to maintain a cylindrical shape. When the retractable wires 47 are disengaged from the occlusion device 10, the medical practitioner may proximally retract the inner catheter 40 from the central lumen 15 of the device 10. In other examples, the inner catheter 40 may be coated with a low friction material, such as MDX or Teflon, so that the inner catheter may be readily detached from the central lumen 15 of the device 10. After the distal ends 20b, 40b of the catheters are detached, the catheters 20, 40 are retracted proximally from the proximal opening 140 and the central lumen 15 of the occlusion device 10. Preferably, the distal end 40b of the inner catheter is retracted proximally into the distal end 20b of the catheter 20 before the catheter 20 is withdrawn from the patient. The occlusion device 10 remains in the left atrial appendage 50. FIG. 7E shows the left atrial appendage 50 in an occluded state 50b with the catheters 20, 40 detached from the occlusion device 10.

After the catheters 20, 40 have been disconnected from the occlusion device 10, before withdrawing the catheters 20,40 the medical practitioner may use the further aperture 26e on the tubular body 22 of the outer catheter 20 to check if the left atrial appendage 50 has been successfully occluded. The distal ends 20b, 40b of the catheters 20, 40 may be positioned near the proximal end 14 of the occlusion device 10. The fluid source 75 (or a separate fluid source, not shown) may be used to deliver contrast 75a through the central lumen 24 of the outer catheter 20 and through the further aperture 26e. The injected contrast 75a may be observed by the medical practitioner using additional imaging to determine whether or not the left atrial appendage 50 is in fluid communication with the left atrium LA, or if the occlusion device 10 has successfully occluded the opening 52 of the left atrial appendage 50. If the injected contrast 75a is observed entering the left atrial appendage 50 (i.e. by moving around the occlusion device 10), then the occlusion device 10 is either filled with more fluid 70, or the occlusion device 10 may be retrieved and a new, larger occlusion device 10 may be deployed into the left atrial appendage 50.

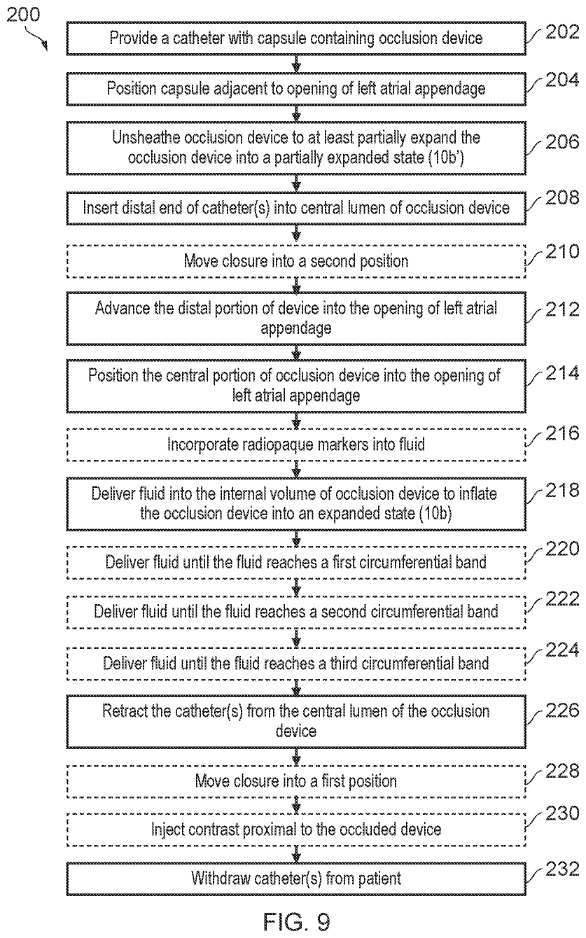

An exemplary occlusion device 10 for occluding a left atrial appendage 50 is described above. An exemplary method 200 for occluding the left atrial appendage is described below in relation to FIG. 9.

At step 202, an occlusion device 10 is provided in a collapsed state 10a inside a capsule 30′ and arranged on the distal ends 20b, 40b of the catheters 20, 40. The distal ends 20b, 40b of the catheters 20, 40 and the capsule 30′ are advanced towards an opening 52 of the left atrial appendage 50 in step 204. At step 206, the occlusion device 10 is unsheathed so that the occlusion device 10 moves from a collapsed position 10a to a partially expanded position 10b′. The occlusion device 10 is unsheathed at step 206 by proximally retracting the capsule 30′ towards the proximal end 20a of the outer catheter 20.

The medical practitioner advances the distal end 40b of the inner catheter 40 through the proximal opening 140 into the central lumen 15 and towards the distal opening 180 of the occlusion device at step 208. Once the distal end 40b of the inner catheter 40 has been extended beyond the distal opening 180, the medical practitioner may optionally secure the inner catheter 40 to the occlusion device 10 by deploying locking wires 47 from the inner catheter. The distal end 20b of the outer catheter 20 is also inserted into the central lumen 15 through the proximal opening 140. If the occlusion device 10 includes a closure 80, advancing the distal ends 20b, 40b of the catheters moves the closure 80 into a second position 80b at step 210.

The occlusion device 10 is advanced into the left atrial appendage 50 at steps 212 and 214. The distal portion 18 of the occlusion device 10 is advanced through the opening 52 of the left atrial appendage 50 and into the internal volume 54 of the left atrial appendage in step 212. At step 214, the central portion 16 is positioned in the opening 52 of the left atrial appendage before the occlusion device 10 is fully expanded by the medical practitioner in the remaining steps. At step 216, radiopaque markers 70a may be incorporated into the fluid 70 before the fluid 70 is delivered into the internal volume 13 of the occlusion device 10 at step 218.

At step 218, fluid 70 is delivered from the fluid source 75 through the central lumen 24 of the outer catheter 20, through the aperture 150 and into the internal volume 13 of the hollow body 12 of the occlusion device 10. The occlusion device 10 is inflated from a partially expanded state 10b′ into a fully expanded state 10b at step 218.

If the occlusion device 10 includes a first, second and third circumferential bands 100a-c, then the medical practitioner may optionally fill the occlusion device 10 to the circumferential bands 100a-c in steps 220-224. At step 220, the medical practitioner delivers fluid 70 into the internal volume 13 of the occlusion device 10 until the fluid 70 reaches the first circumferential band 100a that at least partially extends around the distal end 16b of the central portion 16. At step 222, the medical practitioner delivers fluid 70 into the internal volume 13 of the occlusion device 10 until the fluid 70 reaches the second circumferential band 100b that at least partially extends around the proximal end 16a of the central portion 16. At step 224, fluid 70 is delivered into the internal volume 13 of the occlusion device 10 until the fluid 70 reaches the third circumferential band 100c that at least partially extends around the proximal portion 14 of the occlusion device 10. The steps 220-224 may be completely omitted by the medical practitioner, or alternatively, any number of the steps 220-224 may be completed in any order by the medical practitioner.

One the occlusion device 10 is in an expanded state 10b, the distal ends 20b, 40b of the catheters 20, 40 are retracted from the central lumen 15 of the occlusion device 10. If the inner catheter 40 has been secured to the outer surface 19 of the occlusion device 10 using locking wires 47, the medical practitioner may retract the locking wires 47 back into the central lumen 44 of the inner catheter 40 before retracting the catheters 20, 40 at step 226. The distal end 20b, 40b is retracted from the opening aperture 140 from the proximal portion 14 of the occlusion device 10 at step 226. If the occlusion device 10 includes a closure 80, retracting the distal ends 20b, 40b of the catheters 20,40 from the occlusion device 10 moves the closure 80 into a first position 80a at step 228.

Optionally, the medical practitioner may inject contrast 75a at step 230 after the distal ends 20b, 40b of the catheters 20, 40 have been proximally withdrawn from the occlusion device 10. Preferably, the contrast 75a is injected in the left atrium LA near the left atrial appendage 50. The medical practitioner may observe if the contrast 75a enters the left atrial appendage 50 to verify if the occlusion device 10 has successfully occluded the left atrial appendage 50. If the contrast 75a does enter the left atrial appendage 50, the occlusion device 10 is filled with more fluid 70, or may be removed from the left atrial appendage 50. The medical practitioner may then insert a new occlusion device 10 that is more suitably sized to occlude the patient's left atrial appendage 50.

In the example shown in FIG. 7F, the contrast 75a is ejected into the left atrium LA after the catheters 20, 40 have been proximally withdrawn from the occlusion device 10. In other examples, the medical practitioner may insert contrast 75a into the internal volume 54 of the left atrial appendage 50 after the device is in an expanded state 10b (not shown). In these examples, the medical practitioner may advance the outer catheter 20 towards the distal opening 180 of the occlusion device after disengaging the inner catheter 40 from the occlusion device 10. The medical practitioner may advance the distal end 20b of the outer catheter 20 beyond the distal opening 180 until the further aperture 26e is in fluid communication with the internal volume 54 of the left atrial appendage 50. The medical practitioner may then eject contrast 75a into the left atrial appendage 50 and observe if the contrast 75a exits the left atrial appendage 50 to verify if the occlusion device 10 has successfully occluded the left atrial appendage 50.

In the example where contrast 75a is injected into the left atrium LA, a leak is observed if the contrast 75a re-enters into the internal volume 54 of the left atrial appendage 50. In the example where contrast 75a is injected into the internal volume 54 of the left atrial appendage 50, a leak is observed if the contrast 75a re-enters the left atrium LA. In both examples, a leak is observed if the contrast 75a passes through a gap between the body 12 of the occlusion device 10 and the opening 52 of the left atrial appendage 50.

Once a leak is observed, the medical practitioner may eliminate the gap by expanding the occlusion device 10 further. The medical practitioner may re-insert the distal ends 20b, 40b of the catheters 20, 40 into the central lumen 15 of the occlusion device 10 before introducing more fluid 70 into the internal volume 13 of the hollow body 12. This further expands the occlusion device 10 so that the body 12 sufficiently abuts against the entire diameter of the left atrial appendage opening 52.

At step 232, the distal ends 20b, 40b of the catheters 20, 40 is withdrawn from the patient. Preferably, the distal end 40b of the inner catheter 40 is retracted inside the outer catheter 20 before the outer catheter 20 is withdrawn from the patient

In the example shown in FIG. 7F and described at step 230 in FIG. 9, the contrast 75a is injected after the catheters 20, 40 have been proximally retracted from the central lumen 15 of the occlusion device 10. In other examples, the contrast 75a may be injected before the catheters 20, 40 are withdrawn from the occlusion device 10. This avoids the medical practitioner having to re-insert the distal ends 20b, 40b of the catheters 20, 40 into the central lumen 15 of the occlusion device 10.

In the example where contrast 75a is injected into the left atrium LA without withdrawing the catheters 20, 40, the fifth aperture 26e is positioned on the tubular body 22 of the outer catheter 20 so that the aperture 26e remains in fluid communication with the left atrium LA when the distal ends 20b, 40b of the catheters 20, 40 are positioned inside the central lumen 15 of the occlusion device 10.

In the example where contrast 75a is injected into the internal volume 54 of the left atrial appendage 50 without withdrawing the catheters 20, 40, the medical practitioner may choose to keep the inner catheter 40 secured to the device 10 with the locking wires 47, or alternatively, may choose to retract the locking wires 47. The medical practitioner then proceeds to advance the outer catheter 20 beyond the distal opening 180 until the further aperture 26e is in fluid communication with the internal volume 54 of the left atrial appendage 50 before injecting contrast 75a.

With regard to the terms “distal” and “proximal” within this description, unless otherwise specified, the terms can reference a relative position of the portions of the delivery catheter system with reference to a medical practitioner and/or a location in the vasculature or heart. For example, “proximal” can refer to a position closer to the medical practitioner of the device or an incision into the vasculature, and “distal” can refer to a position that is more distant from the medical practitioner of the device or further from the incision along the vasculature (e.g., the end of the catheter).

Although the teachings have been described above with reference to one or more preferred embodiments, it will be appreciated that various changes or modifications may be made without departing from the scope as defined in the appended claims.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. An occlusion device for occluding a left atrial appendage, the device comprising:
an expandable hollow body with an internal volume,
wherein the expandable body comprises:
a proximal portion having a first diameter, wherein the proximal portion is arranged to sealingly engage against an opening of the left atrial appendage;
a distal portion having a second diameter, wherein the distal portion is arranged to be positioned inside and at least partially fill the left atrial appendage; and
a central portion extending between the proximal and distal portion, wherein the central portion comprises a waist region with a third diameter, wherein
the third diameter of the waist region is smaller than the first diameter of the proximal portion and the second diameter of the distal portion.

2. The occlusion device of claim 1, wherein the central portion is formed from a woven material.

3. The occlusion device of claim 2, wherein woven material comprises a synthetic polymeric material, such as polyetheretherketone (PEEK).

4. The occlusion device of claim 1, wherein the occlusion device is expandable by the introduction of a fluid into the internal volume of the hollow body.

5. The occlusion device of claim 4, wherein the fluid is a biogel.

6. The occlusion device of claim 5, wherein the biogel comprises radiopaque markers.

7. The occlusion device of claim 1, wherein the central portion comprises a plurality of reinforcement struts that are arranged to strengthen the central portion.

8. The occlusion device of claim 7, wherein the central portion is substantially cylindrical, and the plurality of reinforcement struts extend circumferentially around the central portion.

9. The occlusion device of claim 7, wherein the central portion further comprises at least one barb, wherein the at least one barb is arranged to engage with the left atrial appendage to secure the central portion to the left atrial appendage.

10. The occlusion device of claim 1, wherein the distal portion is formed from a woven material.

11. The occlusion device of claim 1 wherein the proximal portion is formed from a woven material.

12. The occlusion device of claim 1, wherein the central portion comprises a proximal end and a distal end, and wherein
the central portion further comprises at least one circumferential band that at least partially extends around the proximal end of the central portion.

13. The occlusion device of claim 12, wherein the central portion further comprises at least one circumferential band that at least partially extends around the distal end of the central portion.

14. The occlusion device according to claim 13, wherein when the central portion comprises at least one band at the proximal end and at least one band at the distal end, both circumferential bands are substantially symmetrical.

15. The occlusion device of claim 1, wherein the occlusion device further comprises a radiopaque material.

16. The occlusion device of claim 1, wherein the distal portion is substantially domed.

17. The occlusion device of claim 1, wherein the occlusion device further comprises a central lumen extending from a proximal opening in the proximal portion to a distal opening at a distal end of the distal portion, and at least one aperture extending from the central lumen, wherein the at least one aperture is in fluid communication with the internal volume of the expandable hollow body of the occlusion device.

18. The occlusion device according to claim 17, wherein the occlusion device comprises a closure that is moveable between a first and second position;
wherein in a first position, the closure prevents flow of fluid in and out of the central lumen through the proximal opening; and
in a second position, the closure permits flow of fluid in and out of the central lumen through the proximal opening.

19. The occlusion device according to claim 18, wherein the closure device comprises a plurality of leaflets, and wherein the leaflets are moveable between the first and second position.

20. The occlusion device according to claim 19, wherein the plurality of leaflets comprise nitinol.
